SEQUENCE LISTING (14 sequences)

SEQ ID NO: 1 - protein, 750 aa, Thermococcus thioreducens
MLMKVLTNMVKLNQDIIPNEIYLYKIFNKPEDGMNIYKIAYRNHGIVIDPQNRIIATPSELEYSGKFAIEDEISFNELPENYQNRLVLRILRDNGISDHALSRTLQKYRKPKPFGDFEVIPEIRSSVIKHGGDFYLVLHLSHQIRSKKTLWELVGRNKDALRDFLKEHRGTILLRDIASEHKVVYKPIFKRYNGDPDLIEDNSNDVEHWYDYHLERYWNTPELKKEFYKKFGPVDLNQPIILAKPLRQHNRGDLVHLLPQFVVPVYNAEQLNDILASEILEYLKLTSNQRISLLSRLINDIKTNTNIIVSSLTELEANTFDVDLNDMLQVRNADNVKVTLSELEISKTRLFTWMKSRKYPVILPYDIPQKLKKIEKIPVFIIVDSALSRDIQTFAKDEFRYLISSLQKSLSNWVDFPILDIRDKYIFTIDLTSDKDIVNLSIKLVNLMKNAELGLALIATRTKLPNETFDEVKKRLFSVNIISQVVNEATLYKRDKYNESRLNLYVQHNLLFQILSKLGIKYYVLRHKFSYDYIVGIDVTPMKLSHGYIGGSAVMFDSQGYIRKIIPVEIGEQMGESIDMKEFFKDMVVQFGKFGIDLEGKSILILRDGKITKDEEEGLAYISKVFGIKITTFNIVKRHLLRIFANRKLYLRLANSVYLLPHRIKQSVGTPVPLKLSEKRLILDGTITSQEITYNDIFEILLLSELNYGSISADMKLPAPVHYAHKFVRALRKGWRIREELLAEGFLYFV

SEQ ID NO: 2 - protein, 750 aa, synthetic construct
MLMKVLTNMVKLNQDIIPNEIYLYKIFNKPEDGMNIYKIAYRNHGIVIDPQNRIIATPSELEYSGKFAIEDEISFNELPENYQNRLVLRILRDNGISDHALSRTLQKYRKPKPFGDFEVIPEIRSSVIKHGGDFYLVLHLSHQIRSKKTLWELVGRNKDALRDFLKEHRGTILLRDIASEHKVVYKPIFKRYNGDPDLIEDNSNDVEHWYDYHLERYWNTPELKKEFYKKFGPVDLNQPIILAKPLRQHNRGDLVHLLPQFVVPVYNAEQLNDILASEILEYLKLTSNQRISLLSRLINDIKTNTNIIVSSLTELEANTFDVDLNDMLQVRNADNVKVTLSELEISKTRLFTWMKSRKYPVILPYDIPQKLKKIEKIPVFIIVDSALSRDIQTFAKDEFRYLISSLQKSLSNWVDFPILDIRDKYIFTIDLTSDKDIVNLSIKLVNLMKNAELGLALIATRTKLPNETFDEVKKRLFSVNIISQVVNEATLYKRDKYNESRLNLYVQHNLLFQILSKLGIKYYVLRGKFSYDYIVGIDVTPMKLSHGYIGGSAVMFDSQGGIRKIIPVEIGEQMGESIDMKEFFKDMVVQFGGFGIDLGGKSILILRDGKITKDEEEGLAYISKVFGIKITTFNIVKRHLLRIFANRKLYLRLANSVYLLPHRIKQSVGTPVPLKLSEKRLILDGTITSQEITYNDIFEILLLSELNYGSISADMKLPAPVHYAHKFVRALRKGWRIREELLAEGFLYFV

SEQ ID NO: 3 - DNA, 2250 bp, synthetic construct
  source 1..2250: mol_type = other DNA
atgctgatgaaagttctgaccaacatggttaaactgaaccaggacattattccgaacgaaatttatctgtacaagatttttaacaagccggaagacgggatgaacatctataaaattgcctatcgcaaccatggtatcgtgatcgatccgcaaaacagaataattgcaaccccaagcgaactggaatacagcgggaagtttgcaattgaagatgaaattagcttcaacgaactgccggagaattaccaaaatcgtctggtgctgcgtatcctgcgggacaacggtattagcgatcatgcactgtccagaacactgcagaaatatagaaaaccgaaaccgtttggcgatttcgaagtgatcccggaaatccgtagcagtgttattaaacatgggggggatttttacttagtgttacatctgagccaccagattcgtagcaagaaaaccctgtgggaactggttgggcgtaataaggacgcactgcgcgattttctgaaagagcaccgtggtacaattctgctgcgtgatattgcatctgaacataaagttgtgtataagccgatttttaagcgttataatggtgatccggacctgattgaagataatagcaatgatgtagaacactggtatgattatcatttagaacggtattggaacaccccggagctgaaaaaggaattttataaaaagttcggccctgtggatctgaatcagccgattatcctggcaaaaccgctgcgtcagcacaatcgcggtgatctggttcacctgctgccccagtttgttgtgccggtttataatgcagaacagttaaatgatatcctggccagcgaaattctggaatatctgaaacttaccagcaaccagagaattagcttactgagccgtttaatcaatgatattaagacaaacaccaacatcatcgtttctagcctgaccgaattagaagcgaacacctttgatgttgacctgaacgacatgttacaagttagaaatgcagataatgtcaaggtgaccctgagcgaactggagatcagcaaaacccgcctgtttacctggatgaaaagtcggaaatatccggtgattcttccatatgatattccacagaaactgaaaaagatcgaaaaaattccggtttttattattgttgatagcgcactgagccgtgatattcagacctttgcaaaagatgaatttcgttatctgattagcagcctgcagaaaagcctgagcaattgggttgattttccgattctggatattcgtgataaatatatttttaccattgatctgaccagcgataaagatattgttaatctgagcattaaactggttaatctgatgaaaaatgcagaactgggtctggcactgattgcaacccgtaccaaactgccgaatgaaacctttgatgaagttaaaaaacgtctgtttagcgttaatattattagccaggttgttaatgaagcaaccctgtataaacgtgataaatataatgaaagccgtctgaatctgtatgttcagcataatctgctgtttcagattctgagcaaactgggtattaaatattatgttctgcgtgggaaatttagctatgattatattgttggtattgatgttaccccgatgaaactgagccatggttatattggtggtagcgcagttatgtttgatagccagggtgggattcgtaaaattattccggttgaaattggtgaacagatgggtgaaagcattgatatgaaagaattttttaaagatatggttgttcagtttggtgggtttggtattgatctggggggtaaaagcattctgattctgcgtgatggtaaaattaccaaagatgaagaagaaggtctggcatatattagcaaagtttttggtattaaaattaccacctttaatattgttaaacgtcatctgctgcgtatttttgcaaatcgtaaactgtatctgcgtctggcaaatagcgtttatctgctgccgcatcgtattaaacagagcgttggtaccccggttccgctgaaactgagcgaaaaacgtctgattctggatggtaccattaccagccaggaaattacctataatgatatttttgaaattctgctgctgagcgaactgaattatggtagcattagcgcagatatgaaactgccggcaccggttcattatgcacataaatttgttcgtgcactgcgtaaaggttggcgtattcgtgaagaactgctggcagaaggttttctgtattttgtt

SEQ ID NO: 4 - DNA, 45 bp, synthetic construct
  source 1..45: mol_type = other DNA
aaacgacggccagtgccaagcttactatacaacctactacctcat

SEQ ID NO: 5 - RNA, 45 nt, synthetic construct
  source 1..45: mol_type = other RNA
aaacgacggccagtgccaagcttactatacaacctactacctcat

SEQ ID NO: 6 - DNA, 15 bp, synthetic construct
  source 1..15: mol_type = other DNA
tgaggtagtaggttg

SEQ ID NO: 7 - RNA, 15 nt, synthetic construct
  source 1..15: mol_type = other RNA
tgaggtagtaggttg

SEQ ID NO: 8 - DNA, 25 bp, synthetic construct
  source 1..25: mol_type = other DNA
tgaggtagtaggttgtatagtaagc

SEQ ID NO: 9 - DNA, 45 bp, synthetic construct
  source 1..45: mol_type = other DNA
aaacgacggccagtgccaagcttactatacaacctactacctctt

SEQ ID NO: 10 - DNA, 45 bp, synthetic construct
  source 1..45: mol_type = other DNA
aaacgacggccagtgccaagcttactatacaacctactacctcct

SEQ ID NO: 11 - DNA, 45 bp, synthetic construct
  source 1..45: mol_type = other DNA
aaacgacggccagtgccaagcttactatacaacctactacctcgt

SEQ ID NO: 12 - RNA, 45 nt, synthetic construct
  source 1..45: mol_type = other RNA
aaacgacggccagtgccaagcttactatacaacctactacctctt

SEQ ID NO: 13 - RNA, 45 nt, synthetic construct
  source 1..45: mol_type = other RNA
aaacgacggccagtgccaagcttactatacaacctactacctcct

SEQ ID NO: 14 - RNA, 45 nt, synthetic construct
  source 1..45: mol_type = other RNA
aaacgacggccagtgccaagcttactatacaacctactacctcgt

(19) United States
(12) Patent Application Publication
(10) Pub. No.: US 2026/0185065 A1
(43) Pub. Date: Jul. 2, 2026

(54) TTDAGO MUTANT WITH TARGET NUCLEIC ACID CLEAVAGE ACTIVITY AT LOW TEMPERATURES, AND USE THEREOF

(71) Applicant: Hubei University, Wuhan (CN)

(72) Inventors: Lixin Ma, Wuhan (CN); Longyu Wang, Wuhan (CN); Fei Wang, Wuhan (CN); Wanping Chen, Wuhan (CN); Wenqiang Li, Wuhan (CN); Yang Liu, Wuhan (CN)

(21) Appl. No.: 19/433,735

(22) Filed: Dec. 27, 2025

(30) Foreign Application Priority Data
Dec. 31, 2024 (CN) 2024119884652

(51) Int. Cl. C12N 9/22; C12N 15/11

(52) U.S. Cl. CPC C12N 9/22; C12N 15/11; C12N 2310/20

(57) ABSTRACT

The invention discloses TtdAgo mutants with target nucleic acid cleavage activity at low temperatures and use thereof. Compared to wild-type TtdAgo, the TtdAgo mutant comprises mutations at amino acid position 527 and/or position 561 and/or position 593 and/or position 599. Relative to wild-type TtdAgo, these TtdAgo mutants not only exhibit significantly enhanced activity at moderate to low temperatures but also enable cleavage of target RNA, thereby effectively expanding the applicability range of pAgo.

### FIELD OF THE DISCLOSURE

The present disclosure relates to TtdAgo mutants with target nucleic acid cleavage activity at low temperatures, and use thereof.

### STATEMENT REGARDING SEQUENCE LISTING

The sequence listing associated with this application is provided in text format in lieu of a paper copy and is hereby incorporated by reference into the specification. The name of the XML file containing the sequence listing is ZL256477-UPS1.xml. The XML file is 17,227 bytes; is created on Dec. 23, 2025; and is being submitted electronically via patent center.

### BACKGROUND

Argonaute (Ago) proteins are an emerging class of programmable nucleases that utilize small DNA or RNA guide nucleic acids (gDNA or gRNA) to cleave complementary target nucleic acids (target DNA and/or target RNA). Unlike widely used Cas nucleases, the catalytic activity of Agos does not depend on specific motifs (such as PAM or PFS) within the target nucleic acids, offering potential for the development of various Ago-based biotechnologies.

TtdAgo is a prokaryotic Argonaute protein derived from the Thermococcus thioreducens. It can cleave target DNA guided by 5′OH-gDNA and 5′P-gDNA, with optimal activity within a temperature range of 70˜80° C. However, these characteristics of TtdAgo limit its application at lower temperatures (such as room temperature) and its capability as an RNA manipulation tool.

### SUMMARY

The present disclosure provides TtdAgo mutants with target nucleic acid cleavage activity at low temperature, aiming to address the limitations of wild-type TtdAgo in terms of catalytic activity temperature and substrate specificity. This is intended to broaden the application scope of TtdAgo and lay the groundwork for its use in fields such as molecular diagnostics, molecular cloning, and gene editing.

In a first aspect, the present disclosure provides several TtdAgo mutants with target nucleic acid cleavage activity at moderate to low temperature. Compared to the sequence shown in SEQ ID NO.1 (i.e., the amino acid sequence of the wild-type TtdAgo protein), These TtdAgo mutants comprise amino acid mutations at position 527 and/or position 561 and/or position 593 and/or position 599.

In the sequence shown in SEQ ID NO.1, the amino acid at position 527 is histidine (His, H), at position 561 is tyrosine (Tyr, Y), at position 593 is lysine (Lys, K), and at position 599 is glutamic acid (Glu, E). Specifically, in the TtdAgo mutants, an amino acid mutation at position 527 refers to substituting histidine with any amino acid other than histidine; an amino acid mutation at position 561 refers to substituting tyrosine with any amino acid other than tyrosine; an amino acid mutation at position 593 refers to substituting lysine with any amino acid other than lysine; and an amino acid mutation at position 599 refers to substituting glutamic acid with any amino acid other than glutamic acid.

Compared to the wild-type TtdAgo protein, the TtdAgo mutants provided by the present disclosure exhibits robust target nucleic acid cleavage activity across a temperature range of 30-90° C. Particularly notable is its superior activity at lower temperatures, endowing it with the ability to efficiently cleave target nucleic acids under ambient conditions.

Preferably, the TtdAgo mutant is mutated to glycine (Gly, G) at positions 527, 561, 593, and 599, either individually or simultaneously. Specifically, the TtdAgo mutant is any of the following:
TtdAgo_H527G, which, compared to the sequence shown in SEQ ID NO.1, carries a mutation at position 527 to glycine;
TtdAgo_Y561G, which, compared to the sequence shown in SEQ ID NO.1, carries a mutation at position 561 to glycine;
TtdAgo_K593G, which, compared to the sequence shown in SEQ ID NO.1, carries a mutation at position 593 to glycine;
TtdAgo_E599G, which, compared to the sequence shown in SEQ ID NO.1, carries a mutation at position 599 to glycine;
TtdAgo_H527G/Y561G/K593G/E599G, which, compared to the sequence shown in SEQ ID NO.1, carries simultaneous mutations at positions 527, 561, 593, and 599 to glycine.

The mutants TtdAgo_H527G, TtdAgo_Y561G, TtdAgo_K593G, and TtdAgo_E599G are all capable of cleaving target DNA under the guidance of gDNA and exhibit enhanced activity at 37° C. Moreover, compared to single-amino acid mutants, the combined mutant TtdAgo_H527G/Y561G/K593G/E599G demonstrates further improved activity at 37° C. It not only cleaves both target DNA and RNA under the guidance of gDNA but also cleaves target RNA when guided by 5′P-gRNA.

In a second aspect, the present disclosure provides biological materials related to the TtdAgo mutants, including:
(b1) nucleic acid molecules encoding the TtdAgo mutants;
(b2) expression cassettes, vectors, or transformants comprising the nucleic acid molecules described in (b1).

For the nucleic acid molecules, any sequence capable of expressing the TtdAgo mutans in an expression system is suitable. For example, in one embodiment of the present disclosure, a nucleic acid molecule encoding mutant TtdAgo_H527G/Y561G/K593G/E599G is provided. Its nucleotide sequence is shown in SEQ ID NO.3, and the amino acid sequence of the encoded mutant is shown in SEQ ID NO.2.

For the vector, it may be a pET plasmid. For example, in one embodiment of the present disclosure, the nucleic acid molecule encoding the TtdAgo mutant is ligated into pET-28a to obtain a recombinant vector. As for the transformant, it can be obtained by transforming host cells with recombinant vectors. The host cell may be a conventional host cell in the field, provided it can support stable autonomous replication of the recombinant vector and enable effective expression of the nucleic acid molecule encoding the TtdAgo mutant.

In a third aspect, the present disclosure provides a nucleic acid cleavage system, which at least comprises: one or more guide nucleic acids, and the TtdAgo mutant provided by the present disclosure.

Preferably, the guide nucleic acid has a length ranging from 11 to 25 nt; within this range, a length of 13-19 nt is more advantageous, with 15 nt being optimal.

Preferably, the guide nucleic acid is selected from 5′P-gRNA, 5′P-gDNA, or 5′OH-gDNA.

In a fourth aspect, the present disclosure provides the use of the TtdAgo mutant in specifically cleaving target nucleic acids. Specifically, it involves constructing a reaction system containing a guide nucleic acid, divalent metal ions, the TtdAgo mutant, and a target nucleic acid to perform a cleavage reaction. The target nucleic acid is complementary in sequence to the guide nucleic acid, and the TtdAgo mutant specifically cleaves the target nucleic acid under the guidance of the guide nucleic acid.

In the aforementioned application method, the target nucleic acid includes target RNA and/or target DNA. The target RNA may be unstructured, structured, double-stranded RNA, in vitro transcribed RNA, viral genomic RNA, mRNA, or other intracellular RNAs. The target DNA may be single-stranded DNA (ssDNA) or double-stranded DNA.

In the aforementioned application method, “complementary” specifically refers to the following two scenarios:
The target nucleic acid contains a nucleotide sequence that is fully complementary to the guide nucleic acid sequence; or,
The target nucleic acid contains a nucleotide sequence that has one or multiple base mismatches with the guide nucleic acid sequence. The number of mismatches may be 1, 2, 3, 4, or 5, and these mismatches are typically isolated but may also be consecutive.

Preferably, the divalent metal ions are Mn²⁺ and/or Mg²⁺, with Mn²⁺ being more preferred.

Given the ability of the TtdAgo mutants to specifically cleave target nucleic acids, it can be utilized in fields such as gene editing and nucleic acid detection.

In a fifth aspect, the present disclosure provides a kit comprising the TtdAgo mutant provided herein. Depending on the detection requirements, the kit may also include guide nucleic acids with specific sequences, buffers to support the catalytic activity of the TtdAgo mutant, and other components.

The advantages of the technical scheme proposed in the disclosure are:

(1) Compared to the wild-type TtdAgo, which can only effectively cleave target DNA using guide DNA at higher temperatures, the TtdAgo mutant provided herein enhances its cleavage activity at medium to low temperatures, broadens its usable temperature range, and retains its heat-resistant capability, which is beneficial for the storage and transportation of the enzyme.

(2) Compared to other TtdAgo mutants, TtdAgo_H527G/Y561G/K593G/E599G is capable of effectively cleaving target DNA using gDNA at medium to low temperatures, as well as efficiently cleaving target RNA using both gDNA and gRNA, demonstrating broad adaptability.

(3) The present disclosure provides expression vectors encoding the TtdAgo mutants (particularly the mutant TtdAgo_H527G/Y561G/K593G/E599G), as well as compositions, kits, and methods for sequence-specific cleavage and editing of target nucleic acids. This lays the foundation for the application of the TtdAgo mutants in numerous fields, such as molecular diagnostics, molecular cloning, and gene editing.

(4) The TtdAgo mutants can cleave target DNA using guide DNA at 37° C., suggesting its potential for genome editing in vivo. Meanwhile, the mutant TtdAgo_H527G/Y561G/K593G/E599G can cleave target RNA using guide DNA at 37° C., indicating its applicability for RNA editing in vivo. Additionally, TtdAgo_H527G/Y561G/K593G/E599G can cleave target RNA using 5′P-gRNA at 37° C., implying that in vivo RNA editing may be achieved through co-expression of the protein and guide nucleic acids.

(5) The TtdAgo mutants strictly relie on the complementarity between the guide nucleic acid and the target nucleic acid to exert its cleavage activity, thereby avoiding the issue of non-specific “collateral cleavage” associated with CRISPR-related proteins and thus offering superior specificity. Moreover, the pAgo complex formed by the TtdAgo mutant and the guide nucleic acid does not require specific motifs near the target site for recognition and binding, making guide nucleic acid design more convenient and free from site-specific constraints.

### BRIEF DESCRIPTION OF THE DRAWINGS

Accompanying drawings provide a further understanding of embodiments of the disclosure. The drawings form a part of the disclosure and illustrate the principle of the embodiments of the disclosure along with the literal description. Apparently, the drawings in the description below are merely some embodiments of the disclosure. A person skilled in art can obtain other drawings according to these drawings without creative efforts. In the figures:

FIG. 1 shows the SDS-PAGE analysis results of TtdAgo protein and its partial mutants in example 1.

FIG. 2 shows the comparative results of cleavage activity between TtdAgo and its mutants at 37° C. in example 2.

FIG. 3 shows the urea-PAGE analysis results of cleavage products obtained using mutant TtdAgo_H527G/Y561G/K593G/E599G with four different guide nucleic acids to cleave target ssDNA or target RNA in example 2.

FIG. 4 shows the urea-PAGE analysis results of cleavage products obtained using mutant TtdAgo_H527G/Y561G/K593G/E599G under different temperature conditions to cleave target ssDNA or target RNA in example 3.

FIG. 5 shows the urea-PAGE analysis results of cleavage products obtained using mutant TtdAgo_H527G/Y561G/K593G/E599G under different metal ions conditions to cleave target ssDNA or target RNA in example 4.

FIG. 6 shows the urea-PAGE analysis results of cleavage products obtained using mutant TtdAgo_H527G/Y561G/K593G/E599G guided by guide nucleic acids of varying lengths to cleave target ssDNA or target RNA in example 5.

FIG. 7 shows the urea-PAGE analysis results of cleavage products obtained using mutant TtdAgo_H527G/Y561G/K593G/E599G guided by guide nucleic acids with different 5′ end nucleotides to cleave target ssDNA or target RNA in example 6.

FIG. 8 shows the urea-PAGE analysis results of cleavage products obtained using mutant TtdAgo_H527G/Y561G/K593G/E599G under different single-base mismatch conditions to cleave target ssDNA or target RNA in example 7.

### DETAILED DESCRIPTION OF PREFERRED EMBODIMENTS

The invention will be further described in detail in combination with embodiments to make the purpose, technical scheme, and advantages of the invention clear.

Unless otherwise defined, all technical and scientific terms used in the disclosure have the same meanings as those commonly understood by those skilled in the art belonging to the disclosure.

TtdAgo is a prokaryotic Argonaute protein derived from the thermophilic archaeon Thermococcus thioreducens. Preliminary studies have shown that it only exhibits cleavage activity against target DNA under the guidance of gDNA. Although it remains active across a relatively broad temperature range, its activity is extremely low at moderate to low temperatures. To overcome the limitations of wild-type TtdAgo regarding its catalytic temperature range and substrate specificity, the present invention provides a modification strategy.

By resolving the ternary complex structure of TtdAgo bound to guide nucleic acids and target nucleic acids, the inventors discovered that the addition of the target nucleic acids induces the formation of its dimerization. Further analysis of its catalytic mechanism was conducted through structural analysis and biochemical experiments. Similar to most Ago proteins, TtdAgo adopts a bilobal architecture: the PAZ lobe (N-terminal domain, linker L1, and PAZ domain) is connected via linker L2 to the PIWI lobe (MID and PIWI domains). The MID and PAZ domains anchor the 5′ and 3′ ends of the guide strand, respectively. The active center within the PIWI domain structurally resembles the active center of RNase H, with the DEDH catalytic tetrad conferring endonuclease activity to TtdAgo. Generally, enzymes from organisms in different environments maintain similar catalytic rates at their respective physiological temperatures, and the sequence variations associated with these adaptive differences are often located in surface-exposed regions distant from the substrate-binding site, leaving the enzyme's active site structurally undisturbed.

Based on the aforementioned research, the inventors successfully screened TtdAgo mutants that exhibit favorable activity under moderate to low temperature conditions by mutating amino acids in the PIWI domain of the wild-type TtdAgo protein located more than 20 Å away from the active site and substrate-binding regions in the three-dimensional structure. Here, the active site specifically refers to the DEDH catalytic tetrad of the wild-type TtdAgo protein.

The TtdAgo mutants provided by the present disclosure, which exhibits favorable activity under moderate to low temperature conditions, contains mutations at position 527 and/or position 561 and/or position 593 and/or position 599, either individually or simultaneously, compared to the wild-type TtdAgo (amino acid sequence as shown in SEQ ID NO.1). Preferably, in the TtdAgo mutant, the amino acids at position 527 and/or position 561 and/or position 593 and/or position 599 are individually or simultaneously substituted with glycine relative to the wild-type TtdAgo. The presence of surface-exposed glycine residues can enhance structural flexibility and enzyme activity through local effects near the catalytic site or via allosteric mechanisms.

Compared to the wild-type TtdAgo protein, the TtdAgo mutants provided in the present disclosure is capable of cleaving both target DNA and target RNA at moderate to low temperatures, thereby demonstrating potential for application in intracellular RNA and genome editing.

The following examples are provided to illustrate specific embodiments of the invention. It should be noted that the examples described below are illustrative only and are intended to explain the invention, not to limit its scope. Where specific techniques or conditions are not indicated in the examples, they should be carried out according to the techniques or conditions described in the literature in the field, or according to product instructions.

### Example 1

This example provides a method for preparing TtdAgo mutants.

### (1) Determination of Mutation Sites.

Load the structure of the TtdAgo ternary complex into PyMOL software, and design mutation sites based on the following three principles: first, the side chain of the mutation site should be highly exposed on the surface, far from the binding substrate and active site (i.e., >20 Å); second, the mutation site is located in the PIWI domain; and third, mutate the amino acid to glycine.

A DNA fragment encoding the wild-type TtdAgo (amino acid sequence as shown in SEQ ID NO. 1) was synthesized and ligated into pET28a. Thus, the pET28a-TtdAgo plasmid was constructed.

Based on the screened mutation sites, TtdAgo mutants were constructed, including: TtdAgo_H527G (histidine at position 527 mutated to glycine), TtdAgo_K528G (lysine at position 528 mutated to glycine), TtdAgo_Q559G (glutamine at position 559 mutated to glycine), TtdAgo_Y561G (tyrosine at position 561 mutated to glycine), TtdAgo_K593G (lysine at position 593 mutated to glycine), TtdAgo_D597G (aspartic acid at position 597 mutated to glycine), TtdAgo_E599G (glutamic acid at position 599 mutated to glycine), TtdAgo_V625G (valine at position 625 mutated to glycine), and TtdAgo_D684G (aspartic acid at position 684 mutated to glycine).

Further combinatorial mutations were performed on TtdAgo, such as simultaneously mutating histidine at position 527, tyrosine at position 561, lysine at position 593, and glutamic acid at position 599 to glycine, resulting in the mutant TtdAgo_H527G/Y561G/K593G/E599G. The amino acid sequence and nucleotide sequence of the mutant TtdAgo_H527G/Y561G/K593G/E599G are shown in SEQ ID NO. 2 and SEQ ID NO. 3, respectively.

Primers were designed for different sites, and PCR was performed using the pET28a-TtdAgo plasmid as the template, followed by T5 recombination cloning to obtain the corresponding plasmids for each mutant.

### (2) Expression and Purification of TtdAgo and its Mutants.

The target protein was expressed using E. coli and induced with IPTG. Purification was performed via Ni-column affinity chromatography and heparin affinity chromatography, followed by analysis using SDS-PAGE. The eluted target protein with higher purity was selected and concentrated using an Amicon 50K ultrafiltration tube. The concentrated protein was aliquoted, rapidly frozen in liquid nitrogen, and stored at −80° C.

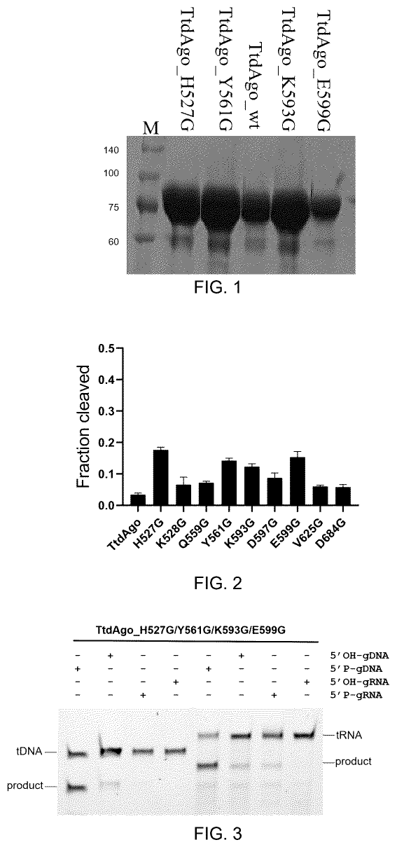

The SDS-PAGE analysis results of TtdAgo and some of its mutants are shown in FIG. 1. The purified TtdAgo and its mutants all have a purity exceeding 90%.

### Example 2

This example tested the cleavage activity of each TtdAgo mutant at low temperatures.

### (1) Cleavage Activity of Wild-Type TtdAgo and its Single-Amino Acid Mutants.

A cleavage test was performed on the target DNA using 5′P-gDNA at a reaction temperature of 37° C.

Add the guide nucleic acid (final concentration 1 μM), TtdAgo or mutant (final concentration 1 μM), and 1× cleavage reaction buffer (10 mM HEPES pH 7.5, 100 mM NaCl, 5% glycerol, 5 mM MnCl₂) into a nuclease-free PCR tube. Incubate at 37° C. for 10 minutes. Then, add 5′FAM-labeled target DNA or RNA (final concentration 200 nM) and react at 37° C. for 30 minutes. Terminate the reaction by adding 2×RNA loading buffer (95% formamide, 18 mM EDTA, 0.025% SDS, and 0.025% bromophenol blue) and heating at 95° C. for 5 minutes. Detect the cleavage products by PAGE. Perform three replicates and analyze the cleavage efficiency using ImageJ and Prism 8 (GraphPad) software.

The cleavage activities of wild-type TtdAgo and its mutants are shown in FIG. 2. TtdAgo_H527G, TtdAgo_Y561G, TtdAgo_K593G, and TtdAgo_E599G exhibit relatively high activity at 37° C.

### (2) Cleavage Activity of Mutant TtdAgo_H527G/Y561G/K593G/E599G.

At 37° C., cleavage tests were performed on target DNA and target RNA using four different guide nucleic acids (5′OH-gDNA, 5′P-gDNA, 5′OH-gRNA, and 5′P-gRNA).

The results are shown in FIG. 3. At 37° C., the mutant TtdAgo_H527G/Y561G/K593G/E599G can cleave target DNA and target RNA using 5′OH-gDNA and 5′P-gDNA, and can also cleave target RNA using 5′P-gRNA.

The sequences of the guide nucleic acids and target nucleic acids used in this example are shown in Table 1.

TABLE 1

| Name | Sequence (5′-3′) |
|---|---|
| FAM-T- | FAM-AAACGACGGCCAGTGC |
| tDNA | CAAGCTTACTATACAACCTA |
| | CTACCTCAT |
| | (SEQ ID NO. 4) |
| FAM-U- | FAM-AAACGACGGCCAGUGC |
| tRNA | CAAGCUUACUAUACAAC |
| | CUACUACCUCAU |
| | (SEQ ID NO. 5) |
| T-gDNA | TGAGGTAGTAGGTTG |
| | (SEQ ID NO. 6) |
| U-gRNA | UGAGGUAGUAGGUUG |
| | (SEQ ID NO. 7) |

### Example 3

This example tested the effect of temperature on the cleavage activity of the mutant TtdAgo_H527G/Y561G/K593G/E599G.

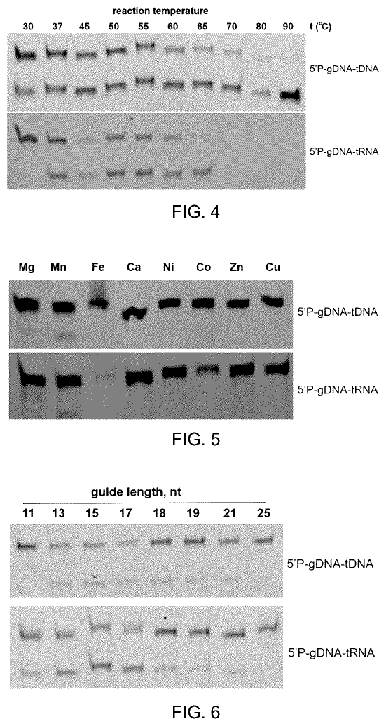

Referring to example 2, cleavage tests of target DNA and RNA guided by 5′P-gDNA were performed with the mutant TtdAgo_H527G/Y561G/K593G/E599G at different temperatures. The results are shown in FIG. 4. The mutant TtdAgo_H527G/Y561G/K593G/E599G exhibits cleavage activity between 30° C. and 90° C., with optimal cleavage efficiency observed between 50° C. and 65° C.

### Example 4

This example tested the effect of divalent metal ion types on the cleavage activity of the mutant TtdAgo_H527G/Y561G/K593G/E599G.

The tests followed that described in Example 2, with the following differences: the guide nucleic acid had a length of 18 nt, and MnCl₂ in the cleavage reaction buffer was replaced with FeCl₂, CoCl₂, NiCl₂, CuCI₂, ZnCl₂, CaCl₂), or MgCl₂ at the same concentration.

The results are shown in FIG. 5. When using 5′P-gDNA to cleave target DNA, mutant TtdAgo_H527G/Y561G/K593G/E599G can effectively cleave the target DNA in cleavage reaction buffer containing Mg²⁺ or Mn²⁺, with superior cleavage efficiency observed in the presence of Mn²⁺. When using 5′P-gDNA to cleave target RNA, mutant TtdAgo_H527G/Y561G/K593G/E599G can also effectively cleave the target RNA in cleavage reaction buffer containing Mg²⁺ or Mn²⁺, with superior cleavage efficiency observed in the presence of Mn²⁺. Thus, the most suitable divalent metal ions for the mutant TtdAgo_H527G/Y561G/K593G/E599G is Mn²⁺.

### Example 5

This example tested the optimal length of guide nucleic acids most suitable for the mutant TtdAgo_H527G/Y561G/K593G/E599G.

Based on the target nucleic acids listed in table 1, a series of gDNAs with lengths ranging from 11 to 25 nt were synthesized. Among these, the sequence of the 25 nt gDNA is shown in SEQ ID NO. 8. The sequences of the 11, 13, 15, 17, 18, 19, and 21 nt gDNAs correspond to the first 11, 13, 15, 17, 18, 19, and 21 base sequences of 25 nt gDNA, respectively. Since the 5′ end sequences of these gDNA are identical, the cleavage sites and the size of the cleavage products are expected to be consistent.

The above gDNAs of different lengths were phosphorylated at the 5′ end, and cleavage tests were performed using these 5′P-gDNAs of varying lengths, following the method described in example 2. The results are shown in FIG. 6. The mutant TtdAgo_H527G/Y561G/K593G/E599G can cleave target DNA using 5′P-gDNA with lengths ranging from 13 to 25 nt, exhibiting the highest cleavage activity with 15 nt 5′P-gDNA. Additionally, the mutant can cleave target RNA using 5′P-gDNA with lengths ranging from 11 to 25 nt, showing relatively high cleavage activity with 15 nt 5′P-gDNA.

### Example 6

This example tested the effect of the nucleotide type at the 5′ end of the guide nucleic acid on the cleavage activity of the mutant TtdAgo_H527G/Y561G/K593G/E599G.

Based on the guide nucleic acids in table 1, this example designed and synthesized a series of 5′ end phosphorylated gDNAs with different 5′ terminal nucleotides (A, T, G, C) but identical sequences elsewhere. Correspondingly, a series of target DNAs and target RNAs complementary to each of the above gDNAs were also synthesized, with specific sequences as shown in table 2.

TABLE 2

| Name | Sequence (5′-3′) |
|---|---|
| FAM-A- | FAM-AAACGACGGCCAGT |
| tDNA | GCCAAGCTTACTATACAA |
| | CCTACTACCTCTT |
| | (SEQ ID NO. 9) |
| FAM-T- | FAM-AAACGACGGCCAGT |
| tDNA | GCCAAGCTTACTATACAA |
| | CCTACTACCTCAT |
| | (SEQ ID NO. 4) |
| FAM-G- | FAM-AAACGACGGCCAGT |
| tDNA | GCCAAGCTTACTATACAA |
| | CCTACTACCTCCT |
| | (SEQ ID NO. 10) |
| FAM-C- | FAM-AAACGACGGCCAGT |
| tDNA | GCCAAGCTTACTATACAA |
| | CCTACTACCTCGT |
| | (SEQ ID NO. 11) |
| FAM-A- | FAM-AAACGACGGCCAGU |
| tRNA | GCCAAGCUUACUAUACAA |
| | CCUACUACCUCUU |
| | (SEQ ID NO. 12) |
| FAM-U- | FAM-AAACGACGGCCAGU |
| tRNA | GCCAAGCUUACUAUACAA |
| | CCUACUACCUCAU |
| | (SEQ ID NO. 5) |
| FAM-G- | FAM-AAACGACGGCCAGU |
| tRNA | GCCAAGCUUACUAUACAA |
| | CCUACUACCUCCU |
| | (SEQ ID NO. 13) |
| FAM-C- | FAM-AAACGACGGCCAGU |
| tRNA | GCCAAGCUUACUAUACAA |
| | CCUACUACCUCGU |
| | (SEQ ID NO. 14) |

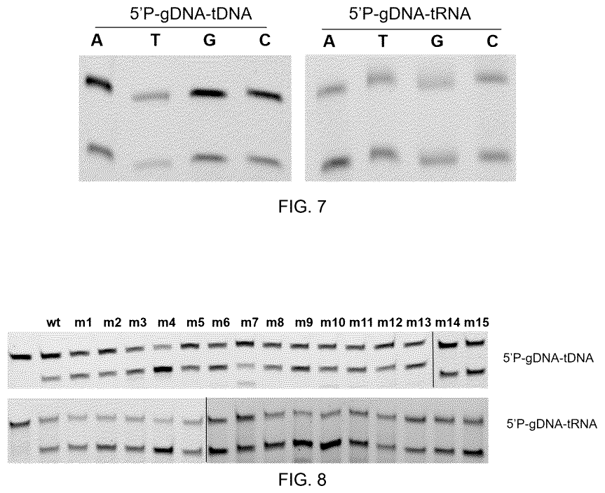

Referring to the method described in example 2, the cleavage activity of the mutant TtdAgo_H527G/Y561G/K593G/E599G on the corresponding target DNA or target RNA was tested under the action of 5′P-gDNA with different 5′ end nucleotides. The results, as shown in FIG. 7, indicate that when the mutant TtdAgo_H527G/Y561G/K593G/E599G cleaves target DNA or target RNA with 5′P-gDNA, no significant preference for specific 5′ end nucleotides was observed.

### Example 7

This example tested the effect of guide nucleic acids containing single-base mismatche on the cleavage activity of TtdAgo_H527G/Y561G/K593G/E599G.

Based on the gDNA, gRNA, and target nucleic acids listed in table 1, a series of gDNA/gRNA were designed and synthesized with single-base difference at various positions compared to the original gDNA/gRNA, resulting in single-base mismatche between the obtained guide nucleic acids and the target DNA or target RNA. Specifically, the sequences of the gDNA/gRNA containing single-base mismatche are shown in table 3.

TABLE 3

| Name | Mismatch Site Information |
|---|---|
| gDNA_mm1 | Replace the 1st position in the T-gDNA with A |
| gDNA_mm2 | Replace the 2nd position in the T-gDNA with C |
| gDNA_mm3 | Replace the3rd position in the T-gDNA with T |
| gDNA_mm4 | Replace the 4th position in the T-gDNA with C |
| gDNA_mm5 | Replace the 5th position in the T-gDNA with C |
| gDNA_mm6 | Replace the 5th position in the T-gDNA with A |
| gDNA_mm7 | Replace the 7th position in the T-gDNA with T |
| gDNA_mm8 | Replace the 8th position in the T-gDNA with C |
| gDNA_mm9 | Replace the 9th position in the T-gDNA with A |
| gDNA_mm10 | Replace the 10th position in the T-gDNA with T |
| gDNA_mm11 | Replace the 11th position in the T-gDNA with C |
| gDNA_mm12 | Replace the 12th position in the T-gDNA with C |
| gDNA_mm13 | Replace the 13th position in the T-gDNA with A |
| gDNA_mm14 | Replace the 14th position in the T-gDNA with A |
| gDNA_mm15 | Replace the 15th position in the T-gDNA with C |
| gRNA_mm1 | Replace the 1st position in the U-gRNA with A |
| gRNA_mm2 | Replace the 2nd position in the U-gRNA with C |
| gRNA_mm3 | Replace the3rd position in the U-gRNA with U |
| gRNA_mm4 | Replace the 4th position in the U-gRNA with C |
| gRNA_mm5 | Replace the 5th position in the U-gRNA with C |
| gRNA_mm6 | Replace the 5th position in the U-gRNA with A |
| gRNA_mm7 | Replace the 7th position in the U-gRNA with U |
| gRNA_mm8 | Replace the 8th position in the U-gRNA with C |
| gRNA_mm9 | Replace the 9th position in the U-gRNA with A |
| gRNA_mm10 | Replace the 10th position in the U-gRNA with U |
| gRNA_mm11 | Replace the 11th position in the U-gRNA with C |
| gRNA_mm12 | Replace the 12th position in the U-gRNA with C |
| gRNA_mm13 | Replace the 13th position in the U-gRNA with A |
| gRNA_mm14 | Replace the 14th position in the U-gRNA with A |
| gRNA_mm15 | Replace the 15th position in the U-gRNA with C |

The above gDNA/RNA strands were phosphorylated at the 5′ end, and cleavage tests were performed following the method described in example 2, followed by detection of the cleavage products.

The results are shown in FIG. 8. When cleaving target DNA guided by 5′P-gDNA, a mismatch at the 7th position (seed region) of the gDNA reduced the cleavage activity of the mutant TtdAgo_H527G/Y561G/K593G/E599G. In contrast, mismatches in the 5′ anchor region, central region, and 3′ supplementary region of the gDNA did not significantly decrease cleavage activity; in fact, a mismatch at the 4th position (central region) even enhanced the mutant's cleavage activity. When cleaving target RNA guided by 5′P-gDNA, mismatches at the 6th or 7th position (seed region) of the gDNA reduce the cleavage activity of the mutant TtdAgo_H527G/Y561G/K593G/E599G. In contrast, mismatches occurring in the 5′ anchor region, central region, or 3′ supplementary region of the gDNA do not significantly decrease its cleavage activity; instead, some mismatches even enhance cleavage activity.

In summary, compared to the wild-type TtdAgo protein, the TtdAgo mutants not only exhibit good cleavage activity at low temperatures but are also capable of cleaving target RNA, effectively expanding the applicability of pAgo. This disclosure further investigated the influence of factors such as gDNA/RNA and divalent metal ions on the cleavage activity of TtdAgo mutants, providing support for their application in various fields such as molecular diagnostics, molecular cloning, and RNA and genome editing.

The above descriptions are only preferred embodiments of the present invention and are not intended to limit the present invention. Any modification, equivalent replacement, improvement, etc., made within the spirit and principle of the present invention shall be included in the protection of the present invention.

What is claimed is:

1. A TtdAgo mutant with target nucleic acid cleavage activity at low temperatures, wherein the TtdAgo mutant is TtdAgo_H527G/Y561G/K593G/E599G,
TtdAgo_H527G, TtdAgo_Y561G, TtdAgo_K593G, or TtdAgo_E599G;
TtdAgo_H527G/Y561G/K593G/E599G, which simultaneously mutates the 527th, 561th, 593th, and 599th positions of the amino acid sequence shown in SEQ ID NO.1 to glycine;
TtdAgo_H527G, which mutates the 527th position of the amino acid sequence shown in SEQ ID NO.1 to glycine;
TtdAgo_Y561G, which mutates the 561th position of the amino acid sequence shown in SEQ ID NO.1 to glycine;
TtdAgo_K593G, which mutates the 593th position of the amino acid sequence shown in SEQ ID NO.1 to glycine;
TtdAgo_E599G, which mutates the 599th position of the amino acid sequence shown in SEQ ID NO.1 to glycine.

2. A nucleic acid molecule, wherein the nucleic acid molecule encods the TtdAgo mutant according to claim 1.

3. An expression cassette, wherein the expression cassette comprises the nucleic acid molecule according to claim 2.

4. An expression vector, wherein the expression vector comprises the nucleic acid molecule according to claim 2.

5. A transformant, wherein the transformant comprises the nucleic acid molecule according to claim 2.

6. A nucleic acid cleavage system, wherein the nucleic acid cleavage system comprises one or more guide nucleic acids and the TtdAgo mutant according to claim 1.

7. A method, wherein the method utilizes the TtdAgo mutant according to claim 1 to specifically cleave the target nucleic acid.

8. The method according to claim 7, wherein the method comprises following steps: constructing a mixture containing guide nucleic acids, divalent metal ions, the TtdAgo mutant, and the target nucleic acid for reaction; the target nucleic acid is complementary to the sequence of the guide nucleic acids, and the TtdAgo mutant specifically cleaves the target nucleic acid under the guidance of the guide nucleic acids.

9. The method according to claim 7, wherein the type of divalent metal ions is Mn²⁺ and/or Mg²⁺.

10. A kit, wherein the kit comprises the TtdAgo mutant according to claim 1.
